(19) United States
(12) Patent Application Publication
(10) Pub. No.: US 2011/0189302 A1
(43) Pub. Date: Aug. 4, 2011

(54) ELECTROCHEMICAL DEVICE

(75) Inventors: David-Lee Van Niekerk, Clonmel (IE); Edmond O'Reilly, Clonmel (IE); Kevin Keane, Kilmacthomas (IE); Eoin Croke, Mooncoin (IE); Michael Campion, Thurles (IE); Brendan Kennedy, Kilmacow (IE)

(73) Assignee: TRUSTWATER LTD., Tipperary (IE)

(21) Appl. No.: 13/056,552

(22) PCT Filed: Jul. 29, 2009
(86) PCT No.: PCT/EP2009/059832

§ 371 Date: Apr. 22, 2011

(30) Foreign Application Priority Data
Jul. 29, 2008 (IE) S2008/0637
Jul. 29, 2008 (IE) S2008/0638
Jul. 29, 2008 (IE) S2008/0639

(51) Int. Cl. C25B 15/00; C25B 15/08; A61K 33/00; A01P 1/00

(52) U.S. Cl. 424/600; 204/237; 204/229.8; 204/275.1

(57) ABSTRACT

There is provided an automated electrochemical device for generating a biocidal output solution, said device comprising: a flow-through electrochemical cell comprising an anodic chamber and a cathodic chamber for electrolysing an electrolyte to generate an anolyte solution and a catholyte solution; characterised in that the device further comprises: (i) a reservoir for storing catholyte; and (ii) a hydraulic circuit for recirculating catholyte from the reservoir to the anolyte on start-up of the cell, wherein input of catholyte of a compensating strength to the cell anodic chamber, is arranged so as to optimise the cell anolyte pH to produce a stable output solution at the start of the electrolysis process.

### FIELD OF THE INVENTION

The invention relates to improved electrochemical devices, more particularly, to electrochemical devices comprising a flow-through electrochemical cell (FEM), and electrolysis of solutions therein. In particular, the invention relates to aqueous solutions, for example, aqueous brine or other ionic salt solutions, of suitable concentrations and pH to produce anolyte and biocidal catholyte output streams when electrolysed in such electrochemical devices.

### BACKGROUND TO THE INVENTION

In the field of applied electrochemistry, chemical electrolysis generally occurs in an electrochemical cell, wherein an electric current is passed through either a solution of a solvated, commonly aqueous, ionic substance or a molten ionic substance. Electrolysis processes produce new chemical species, which can subsequently take part in chemical reactions at the cell cathode and anode to form new compounds.

A common electrochemical process involves the electrolysis of aqueous sodium chloride (or brine) solutions in a diaphragm cell. A diaphragm cell is of a type, where the cell is divided by an ion permeable membrane or separator into anodic and cathodic chambers. Chlorine, hydrogen gas and sodium hydroxide are the primary products produced by this particular electrolysis system, though small amounts of ozone, peroxide and chlorine dioxide can also be formed, depending on the configuration of the cell. In such a cell chloride ions migrate to, and are oxidized at, the anode in the anodic chamber to form chlorine atoms. These chlorine atoms react together to form chlorine gas, the process summarised by the following half reaction,

2Cl⁻→Cl₂+2e⁻.

Water molecules are reduced at the cathode to form hydroxyl anions and hydrogen gas in the cathodic chamber. Solution sodium ions migrate to the negative cathode where they can interact with hydroxyl ions produced at the cathode and thus constitute the components of sodium hydroxide formed during the electrolysis of brine. Thus, as the cathodic reaction proceeds, the pH of the solution in the cathodic chamber increases and the solution becomes increasingly basic (catholyte) as hydroxide concentration increases.

The chlorine produced dissolves and reacts with water producing hypochlorous acid and hydrochloric acid.

Cl₂+H₂O→HOCl+HCl.

As the solution becomes acidic, this reaction is less favoured and the chlorine instead dissolves in water without undergoing subsequent hydrolysis. However the solubility of chlorine in water is limited and off gassing of chlorine will occur once this threshold is exceeded. At higher pH values, an equilibrium between hypochlorous acid and hypochlorite ion is established with an acid dissociation constant of 7.5. It is clear is that the pH of the solution is of critical importance and will have a fundamental effect on the solution species “free chlorine” equilibrium concentrations and states.

Electrolysis of water also occurs at the anode, according to the following reaction:

2H₂O→4H⁺+O₂+4e⁻.

Oxygen gas is liberated and hydrogen ion production results in the pH of the anode solution (anolyte) falling to become acidic. This reaction is undesirable, as it reduces cell efficiency in terms of chlorine production and is inhibited and minimised in an acidic electrolyte environment.

A particularly useful application of typical brine chemical electrolysis involves generation of powerful biocide solutions comprising the strong oxidant hypochlorous acid. Such biocidal solutions are valuable in applications involving disinfection and sanitisation of water, surfaces, processing equipment and also finds use in food processing. The solutions are typically biocidal against many species such as bacteria, viruses and fungi, etc. However, an associated shortcoming with existing biocide solutions is the often large variation in the pH, salt concentration and available “free chlorine” found in such solutions. The variation in solution composition will depend on, for example, the condition of inputs to the electrochemical cell and variations in the current, temperature and/or pH across the cell as electrolysis proceeds.

Free chlorine based biocide solutions are generally composed of one or more of dissolved chlorine, hypochlorous acid and hypochlorite ion depending on pH, but can also contain varying amounts of other species including, for example, ozone and chlorine dioxide. In addition byproducts such as chlorate can be produced, one way of forming which, is by reaction of hypochlorus acid and hypochlorite ion. Although it is known that free chlorine is an effective biocide it is true to say that the precise mechanism of biocidal action is not yet fully appreciated.

Solutions of free chlorine solutions can be corrosive due to their elevated Oxidation Reduction Potentials (ORP). This problem is most acute for free chlorine solutions that also contain high concentrations of chloride ion. Solutions of free chlorine always contain a certain amount of chloride ion, which promotes the particularly vigorous pitting form of corrosion, due to the nature of the hydrolysis reaction between chlorine and water. The amount of chloride ion released into the water by this reaction is typically not problematic. However many methods and devices for the electrochemical production of free chlorine solutions are characterised by poor conversion of chloride ion to free chlorine and the chloride ion concentrations in the biocide attained using these devices can be of serious concern. It is therefore desirable that an electrochemical device should be efficient at converting chloride into free chlorine so that operating costs and corrosion problems are minimised.Existing methods of producing biocidal solutions that are 10-400 ppm “free chlorine” involve electrolysis of low salt concentration solutions (1 to 3 g/L) using current densities below 1 kAm⁻². Great Britain Patent No. 2 352 728 describes the electrolysis of a solution of 3 to 5 g/L sodium chloride using a current of 7 to 9 A (current density 0.8 kAm⁻²) in the production of biocidal solutions of 100 to 400 ppm “free chlorine”. European Patent No. 0 832 850 discloses a process of electrolysing dilute brine solutions, however no specific current density information is provided. Flow rates are high (250 L/hr), the only output parameters that control the biocide output specification are pH and redox potential. European Patent No. 838 434 and International Publication No. WO 98/12144, all describe the preparation of biocidal solutions by the electrolysis of a concentrated or saturated (up to 250 g/L) solution of sodium chloride to form chlorine gas, which is then dissolved in water to produce the biocidal solution. European Patent No. 0 792 584 describes a method for the preparation of biocidal solutions of pH 3 or less and hypochlorous acid concentrations of about 2 ppm. U.S. Pat. No. 5,731,008 describes the preparation of biocides having an active chlorine species content between 10 and 100 ppm.

A multitude of electrolysis cells of varying type, function and design are available. One design of a typical biocidal output producing electrochemical cell consists of two concentrically disposed cylindrical electrodes with an ion permeable membrane separating the space between the two electrodes. The diaphragm arrangement has the effect of defining the anode and cathode chambers and substantially isolating them from each other. The resulting solutions are restricted from mixing by the membrane separator. European Patent No. 0 842 122 describes a flow-through electrolytic module (FEM) which produces a biocide solution. Such FEMS are of interest within the context of the present invention.

Current passing through the FEM cell results in generation of anodic and cathodic products in their respective chambers. The overall rate of an electrochemical reaction is proportional to the current flowing through the cell, and so the rate of cell electrolysis can be adjusted by varying the current passing through the cell. A higher current means faster electron flow and higher rate of electrolysis. In general, control of cell current will produce a biocide output having a desired concentration of biocidal components.

The hydraulic systems of electrochemical cells are critical to automated cell operation and allow automated devices to operate efficiently, and thus make biocidal solution production more commercially viable. There are many existing designs for the hydraulics systems of electrochemical generators. One existing design attempts to compensate for fluctuations in cell current, and thus cell output, by measuring the cell electrolyte solution concentration over time and making adjustments, if necessary. Existing systems do not function that well in this regard and a certain degree of output fluctuation is unavoidable.

Improved designs deal with this problem by allowing for the discharge of electrolytic saline solution into the anode chamber, if such solution adjustment is required. This is useful in cases where the electrochemical cell voltage is fixed, since the amount of ionic material present (which may depend on the amount of solution and the concentration of that solution in the anode chamber), will in part determine the total current in the cell. Generally, the amount of saline solution in such cells is controlled by monitoring alterations in upper and lower saline cell level (height of solution in the cell) limits. Thus, the cell current is adjusted by measuring the electrolyte level in the electrochemical cell. More specifically, when required, saline solution can be discharged into the cell until the upper electrolyte level limit is reached, at which time discharge ceases. This has the effect of increasing the number of ions available in the cell and as a result, the current flowing through the cell. The electrolysis process then continues until the saline solution in the anode chamber reaches the lower electrolyte level limit, at which point more solution is discharged into the anode chamber to bring the cell once again to the upper level limit.

It should be pointed out that such a replenishing process has the effect of (i) causing the cell current to steadily rise, when fresh saline solution is discharged into the cell and (ii) allowing the current to steadily decrease as electrolysis of the salt solution takes place. Since the output from the cell is a function of the current passing through the cell, the effect results in the gradual decline of the current in the cell as processing is taking place, resulting in a gradual decline in the concentration of the output solution from the cell over the same period. Over an extended period of time, the output solution of the cell decreases accordingly. Thus, the existing method of controlling the current in an electrochemical processing cell is lacking and gives rise to an undesirable and inefficient rise and fall cycle in the output from the cell. In an automated biocide producing system, this effect is unfavourable, since it leads to inconsistent chlorine gas generation and biocide component output variability.

In such cells hydroxide ion is produced at the cathode and it is common that the catholyte is continuously circulated through the cathode chamber. This is advantageous for a number of reasons, but in particular, recirculation allows for heat exchange to occur between the cell and the catholyte and so allows control of the electrochemical cell temperature. This is important, since temperature will affect the kinetics of the electrochemical processes in the cell.

Another advantage of continuous catholyte circulation is that the catholyte solution may be dosed into the anolyte solution to modify the pH of the output. Indeed, it is normal practice to discharge some circulating basic catholyte solution from the device into the new anolyte solution to achieve the desired pH. However, alkaline catholyte solution is corrosive and can damage the electrochemical cell and hydraulics, if it remains in the cell when the electrolysis is not taking place. As a result, the catholyte is drained from the cell when the device is shut down.

Many applications for the output solutions and particularly the biocide outputs from an electrochemical device are pH sensitive. Indeed, the final pH of a biocide output is very important, since unstable pH variations will have an effect on the concentrations and equilibrium species present in the final solution, affecting the biocidal properties. Currently, it is normal that during the start up period, the initial output from the device is not suitable for commercial use until such time as the output is produced at the desired pH and that the pH is sufficiently stable (ensuring that the required species are present in the desired equilibrium concentrations). When an electrochemical device starts initial processing of the electrolyte solution in the catholyte chamber, the ensuing catholyte solution has an unoptimised pH (due to low hydroxide concentration for example in the case of electrolysis of salt electrolytes) that increases as the electrolysis proceeds (i.e., becomes more basic). There is an initial period of time during start up when the hydroxide concentration of the catholyte is low. During this period, the pH of the output biocide from the device cannot be kept stable, since the low hydroxide concentration catholyte produced during the start up period is not of sufficient strength to regulate the output pH of the device. A pH-stabilized output cannot be produced until the catholyte increases sufficiently in strength. Thus, the initial output biocide solution from the device must be discarded until the required catholyte hydroxide concentration is attained. Generally, the effect leads to a long start up period for the device, which result in the initial output being commercially undesirable and wasteful.

A further consideration for the production of a consistent output biocide solution by an electrochemical device is the condition of the inputs. Critically, the inputs must be of a sufficiently high standard so as to allow smooth electrolysis, a consistent output and the continued uninterrupted operation of the electrochemical device. For example, for FEM-based devices, most commonly the inputs to the device are salts, water and electricity. The electricity can readily be conditioned to a standard required for efficient processing. The salts are generally available at a standard that is sufficient for the consistent operation of the device. The water, however, varies dramatically depending on the geographical region, chemicals added and the actual type of conditioning occurring through the devices, for example, filtration, softeners, and treatment by reverse osmosis etc. In general, electrochemical devices are sensitive to contaminants in the supply water. A good example of contamination is that occurring from use of “hard water”, which essentially is water that has a high mineral content. Hard water is usually comprised of calcium, magnesium ions, with possible counterions including bicarbonates and sulphates. Hard water can result in mineral deposits that cause a change to the permeability of the electrochemical generating cell membrane, resulting in decreased efficiency of the cell and eventual failure of the device. Furthermore, on occasion, failure in device operation can result in unsafe conditions for operators, damage to the device itself or to other equipment in the vicinity of the electrochemical device. Consequently, it is desirable to pre-treat or condition the input solutions and chemicals. However, such treatment is generally prohibited by the large costs associated with conditioning of the large volume of solutions required.

A final consideration is the gas pressure produced in the generating cell. Cell gas pressure is a critical controlling parameter that determines the efficiency of the generating process. Gas pressure affects the operation efficiency of the device. Moreover, excessively high pressures may result in damage to the cell semi-permeable membrane and may cause the device to fail. On the other hand, if the pressure is too low, the device will take a long period to commence operating on start up. Importantly, the pressure in the anolyte chamber will also determine the amount of salt in the output solution from the device. It is worth noting that a regular failure mode of existing systems is excessive pressure and temperature in the electrolysis cell which may cause the membrane to leak, crack or break. Present systems maintain gas pressure in the cell in a very crude way, usually in the form of a mechanical pressure regulator or the like. This is undesirable since such regulators are only adjustable manually and do not allow fine control of the system and resulting outputs.

In order to address some of the deficiencies currently associated with the prior art, there is therefore a need to provide improved electrochemical devices, hydraulic systems and device modules for use in such devices wherein the systems facilitate the automated production of a more consistent and stable output biocidal solution.

In particular, it is desirable to provide improved electrochemical devices and hydraulic systems for use in such devices that have the capacity to address the problems outlined above.

### SUMMARY OF THE INVENTION

In one aspect as described herein, the electrochemical device of the invention may be used in the production of biocidal solutions, the device comprising:

(i) an electrochemical cell configured to produce both a gaseous product composed of chlorine in the main and a basic caustic solution (catholyte),

(ii) a control system to regulate the condition of solutions inputted to the cell, the performance of the cell and the production of biocidal solutions of regulated pH (anolyte) from the products of the electrolysis reactions in the cell.

The skilled person will appreciate that the electrochemical cell may any type of cell capable of electrolysing an electrolyte solution. Preferably the cell is a flow through electrochemical cell (FEM), including FEMs of the flat plate or conical type.

In another aspect as described herein, the invention discloses a means to regulate the performance of the cell comprising a current detection system and a method of using such a system, to provide a means for ensuring stable and consistent biocide output from an electrolysis cell. The current detection system is in communication with the control system and measures the total electrochemical current in the cell, since the current detection device is electrically connected to the electrolysis cell. Current measurements are made as the electrolysis reaction proceeds so that throughout the electrolysis process, the current in the cell is monitored. The current measurement data is then used by the control system to calculate the amount of saline/electrolyte solution to be input into the cell from an external reservoir to stabilize any observed changes in cell current. When the current detection system indicates that a current decrease has occurred, saline solution input is initiated to increase and restore the output product concentration stability from the cell.

Accordingly, the current level in conjunction with the level of electrolyte solution in the cell serves as an indicator of the efficiency of the cell. The invention discloses another parameter that may be used for the calibration of cell efficiency namely the measurement of gas pressure in the electrochemical cell.

The device of the invention also provides an automated system which is capable of producing faster system start up times with less wasteful initial outputs by a system of catholyte re-circulation, designed to optimise anolyte pH quickly, by dosing of anolyte with the required amount of catholyte. A stable output pH is critical to producing biocide with the desired properties, since the pH will affect the degree of dissociation of hypochlorus acid formed during electrolysis. Initial component concentrations of the catholyte, at device start-up, are not concentrated enough or sufficiently conductive to regulate the pH of the output solution and ensure that the cell operating current is achieved. Re-circulation has the effect that the pH of the catholyte increases over time as electrolysis proceeds and the sodium hydroxide concentration of the solution increases. However, when an electrolysis device starts up, the catholyte solution initially produced has a low pH due to lack of hydroxide ions in the solution. The low concentration of catholyte components produced during the start up period may not of sufficient strength to ensure that the operating current of the device can be achieved.

Finally, as described herein the invention provides a system and method that allows for reduction of the time it takes to produce the desired catholyte output pH and consequently reduces the time for normal operating currents to be achieved. On start up of the device, the stored catholyte (basic if stored from a previous operation) can be used to mix directly with the anolyte or with the actual output solution, as is required. Thus, the invention discloses an automated system and method of use wherein the catholyte solution is stored in a vessel during device operation and delivered to the electrolysis cell or output stream on system initiation as required to decrease start-up time.

Accordingly, in a first aspect of the invention, as set out below and in the appended claims, there is provided an automated electrochemical device for generating a biocidal output solution, said device comprising:

(i) a flow-through electrochemical cell for electrolysing an electrolyte to generate the output solution;

(ii) a current detecting system connected to the electrochemical cell for determining when cell current reaches a predetermined level; and

(iii) an electrolyte delivery system operable by the current measuring system, characterised in that the electrolyte delivery system inputs a volume of electrolyte into the cell when a predetermined level of current is detected, so that the generated output solution of the electrochemical cell has a substantially constant concentration.

The present invention provides an improved automated electrochemical device capable of automated continuous adjustment to produce a substantially constant output solution having stable component concentrations and/or pH. The automated continuous adjustment may be set up to operate by detecting current periodically over a fixed period which may range from fractions of a second to periods of minutes or longer. The length of the current detection period will be determined by the level of biocidal output consistency required. In some applications the period may be from 1 millisecond to 1 second. In other applications the period may be from 1 second to 60 seconds. In further applications the period may be from 1 minute to every 60 minutes etc. Thus, there is provided an improved automated means of stabilising the current in the electrolysis generating process and a system capable of, and a method for, automatically and continuously adjusting the cell current to provide a substantially stable current which results in production of a consistent output (having consistent levels of biocidal components in the case of a biocidal output).

The automated system and the method of using same, ensures a more stable current output in a fixed voltage electrochemical cell, where traditionally current output is more cyclical. Suitably, this is achieved by use of a control system for continuously controlling input of the additional electrolyte to the electrochemical cell, whereby the control system can act on current data provided by the control system so as to maintain a current passing between the electrodes at a steady state level. This system is advantageous over prior art systems since continuously maintaining a steady state cell current based on current monitoring within the cell will ensure that a more accurate consistent and stable output product solution is generated over an extended period of time whereas existing systems based on monitoring electrolyte level in the cell results in less consistent outputs, particularly since the electrolyte levels are prone to external effect such as temperature and catholyte flow effects within the cell.

In this aspect, the invention discloses an automated electrochemical device comprising a current detection system and a method of using such a system, to provide a means for ensuring stable and consistent biocide output from an electrolysis cell. The current detection system is in communication with the control system and measures the total electrochemical current in the cell, since the current detection device is electrically connected to the electrolysis cell. Current measurements are made as the electrolysis reaction proceeds so that throughout the electrolysis process, the current in the cell is constantly monitored over defined or predetermined desirable intervals of time ranging from fractions of seconds to minutes to hours if desired. The current measurement data is then used by the control system to calculate the amount of saline/electrolyte solution to be input into the cell to stabilize any observed changes in cell current. When the current detection system indicates that a current decrease has occurred, saline solution input is initiated to increase and restore the overall efficiency and output product concentration stability from the cell. The current level in conjunction with the level of electrolyte solution in the cell serves as an indicator of the efficiency of the cell.

Advantageously, the current may be detected, measured, determined and/or calculated by the current detecting system, which may suitably comprises a current measurement detection device and/or an evolved gas pressure measurement device, both of which are under management of a control system. The electrolyte may be any ionic solution, however, for biocidal output solution production, aqueous salt electrolytes may be suitably used. Examples of such aqueous salt electrolytes which will produce the necessary chlorine gas at the FEM anode include aqueous solutions of ionic salts such as NaCl, KCl, LiCl, etc. It is preferred that NaCl salt solutions are used, since NaCl is freely available, is cheap and non-toxic to handle. More preferably still, brine solutions may be used to produce basic catholyte solutions and anolyte solutions containing dissolved chlorine, along with other and more favourable electrochemical products. Chlorine gas is a particularly desirable product within the cell. The anolyte produced from aqueous brine solutions may comprise a mixture of antimicrobial and disinfecting agents, such as dissolved chlorine, hypochlorous acid and hypochlorite ion. They can also contain varying amounts of antimicrobial and disinfecting radicals or ions including, for example, ozone and, chlorine dioxide. The anolyte solution may also contain ionic salts such as NaCl or KCl or combinations of same, depending on the form of the starting ionic salt electrolyte used. The amount of salt in the output depends on the chlorine gas pressure at the anode.

The flow-through electrochemical cell (FEM) is typically a cell separated into an anodic chamber and a cathodic chamber by an ion permeable membrane or suitable separator. It may be of the flat palte or coaxial cell type. Suotable cells include the type described in European Patent No. 0 842 122, the contents of which are incorporated herein by reference. When current is passed through the FEM, the electrolyte solution dissociates and the ions migrate across the FEM membrane to the oppositely charged electrodes, where the appropriate redox reaction occurs. Hydrogen ions and chlorine gas are produced at the anode and dissolved to form an increasingly acidic anolyte solution with time, while hydroxide from the aqueous solution is formed at the cathode to form an increasingly basic catholyte solution as the electrolysis reaction proceeds. The anolyte solution forms the basis for the biocidal output solution. The skilled person will appreciated that the term “by anolyte solution”, it is means that the solution is in fact a composition comprising gas, solution, aerosol or combinations thereof.

Accordingly, in the first aspect, the invention also provides a method of generating a stable biocidal output from an electrochemical cell comprising the steps of:

(i) detecting the current in the cell;

(ii) inputting electrolyte into the cell when a predetermined minimum current level is measured; and

(iii) ceasing input when the current reaches a predetermined maximum level.

Advantageously, the current may be detected, measured, determined and/or calculated by the current detecting system, which may suitably comprises a current measurement detection device and/or an evolved gas pressure measurement device, both of which are under management of a control system. It is preferable that a current measurement device is used, since advantageously, such a device can be used to directly measure the current flowing across the cell. A multimeter may be used to measure the current. However, any electrical measurement device known to the skilled person may be suitably used to calculate or measure the flow of electric current in the cell. Such devices may comprise, but are not limited to, for example, an ammeter, a galvanometer, a multimeter device or the like. However, in the present system, a current transducer is preferred, since it will ensure for accuracy of data. Alternatively, an evolved gas pressure measurement and dynamic adjustment device may be used as the current detection device to provide data to the control system to allow it to calculate the current in the cell. This is possible, since evolved gases at the electrode indicate the degree of electrolysis and hence the current flowing across the cell. Furthermore, if the current is maintained at a substantially constant level, then the data can be used to provide information as to the cell efficiency. Thus the invention provides an improved automated electrochemical device, and a hydraulic system for use in such a device and a method of using same, that facilitates measuring, controlling and adjusting the gaseous pressure in the system so as to allow compensations to be made to the system when required and to allow control of salt formation in the output solution.

Means for detection and control of the gas pressure in cell anode and the hydraulic system is desirable, since it allows adjustment of the gas pressure to a desired level to be made, so that the cell operates efficiently and the salt concentration in the biocide output solutions can be finely controlled. Such adjustments can be at regular intervals or can be dynamic in the sense that adjustment is essentially continuous. Thus detection and adjustment can occur over defined or predetermined desirable intervals ranging from fractions of seconds to minutes to hours if desired or as necessary. Thus, the use of a gas pressure measurement and automated adjustment device is advantageous, since it will also allow the salt content of the output solution to be controlled. The pressure in the anode chamber alone or when used with the current data and/or volume or level measurement, may also be used to determine the efficiency of the electrochemical device. Thus, it is desirable to provide a device and a method whereby the pressure in the hydraulic system can be detected and a control mechanism is put in place that adjusts the pressure to a particularly desired level depending on the levels of salt required for the output solution and the operating parameters of the FEM. It will be appreciated that this may be accomplished by using an electrical pressure valve or any means for dynamic or continuous, automated gas pressure adjustment. It is preferable that the gas pressure data is relayed to the control system that is linked to the gas pressure valve and is controllable there from. The gas pressure can be tracked over time allowing for the continued evaluation of the efficiency of the system. Sudden, dramatic or sustained changes in gas pressure outside the control set points can provide evidence of a decrease in efficiency, failure and/or imminent failure in the system. As efficiency decreases, less gas will be produced and the gas pressure will gradually drop over time. A gas pressure meter installed in the device can be set to signal a warning on reaching a lower gas pressure limit. Once a change in efficiency is detected, the information can be used for a number of purposes, for example, to initiate an error or warning notification to the operator that efficiency has changed or to produce a signal to stop the device, initiate a cleaning process or schedule a device service. The gas pressure measurement device may also be used to determine the amount of salt in the output stream and thus a gas pressure regulator serves as a useful means to control salt concentration in the output by changing the gas pressure at the electrodes. Thus, the pressure in the generating cell is an important parameter determining the current and/or the efficiency of the generating process and the levels of salt in the output solution, since the pressure is an indicator of the amount of chlorine gas produced at the anode of the cell and so provides a measure of cell performance and efficiency over time.

Thus, the device is advantageous in that electrochemical cell operating efficiency compensations and output component concentrations can be accomplished by monitoring system variables such as evolved gas pressure, cell current and cell electrolyte volume or height level and making electrolyte input adjustments or cell anode gas pressure adjustment accordingly. Thus, monitoring at least one of these parameters, allows the control system to compensate for loss of efficiency by increasing the input of fresh electrolyte. As the efficiency of the cell decreases, incremental amounts of fresh electrolyte will have to be input to the system to maintain a constant current passing through the cell. This system is useful since the cell can be set-up such that when a preset amount of compensating input is reached, the control system will cease to input further electrolyte until such time the cell is serviced, cleaned or otherwise treated to restore the gas pressure, cell current and consequently the cell efficiency to a previous state.

Thus devices incorporating some or all of the features capable of indicating and automatically adjusting gas pressure are advantageous, since in addition to regulating efficiency and output salt concentrations, they facilitate shorter start-up times by adjusting low pressures and avoid build up of excessive pressure in the device which can result in membrane damage such as cracking, breaking or leaking.

In a related embodiment there is provided a method of controlling the salt concentration in an electrochemical cell output comprising the steps of:

(i) measuring the evolved gas pressure in the cell; and

(ii) adjusting the gas pressure to a predetermined level, so that the salt concentration in the output is within a predetermined range.

As discussed earlier, the system can be modified to alert the operator that the system requires attention. The skilled person will appreciate that the efficiency of the electrochemical device can be determined by measuring one or more of a number of variables, for example, the cell current compensation required over time and/or the corresponding volume or level of anolyte solution in the FEM anodic chamber or variations in the gas pressure at the electrodes. Thus, the level and or volume of solution in the anode chamber combined with the current level and/or gas pressure at the anode, may also be used to determine the efficiency of the electrochemical device. In addition to volume measurement means, the cell may be fitted with a electrolyte visualising means, for example, a transparent area comprising glass or the like, which will allow the level of the electrolyte in the cell to be directly observed. The area may be calibrated to indicate particular level(s) that represent particular degrees of efficiency loss. The system can also be calibrated to account for temperature and catholyte flow effects in the cell and their effect on anode liquid level. The operator may then visualise efficiency decreases over time and provide them with notice that the critical loss of efficiency is pending. The level and or volume of solution in the cell may also be detected with an automated device (for example a level sensor) allowing for a fully automatic detection of cell efficiency. Depending on the system, the information may be relayed to the control system so that appropriate remedial action may be promptly taken.

There is further provided an improved electrochemical device, and a hydraulic system for use in such a device and a method of using same wherein at least one of: the change in cell current over time, the gas pressure at the electrodes and/or the volume and/or level of electrolyte present in the cell when the current is stable, or changes therein over time, can provide information as to the overall efficiency of the cell over when monitored over a set period of time. Thus, there is provided a method of determining the efficiency of an electrochemical cell comprising at least one of:

(i) measuring the change volume of electrolyte input required to maintain a substantially constant cell current over a set period of time; or

(ii) measuring the change in the cell gas pressure over time, when the cell current is substantially stable; or

(iii) measuring at least one of, the volume or the level of electrolyte in the cell over time, when the cell current is substantially stable;

wherein measuring a predetermined change in, the volume electrolyte input or cell gas pressure or change in volume or height of electrolyte in the cell over a set period of time results in identifying a critical loss of cell efficiency.

As discussed earlier, the electrolyte solutions are generally aqueous solutions of ionic salts such as NaCl, KCl, LiCl etc. NaCl solutions are suitably preferred. Furthermore, dilute solutions of such ionic electrolytes are particularly preferred, for example, brine. However, in certain applications concentrated saline solutions are most preferred. Rock salt, sea salt, or refined salt (table salt) may equally well be used, as may some other mineral compositions high in NaCl. Thus, saline solution may be suitably used as the electrolyte. Such solutions may be preferably discharged into the electrochemical cell though an electrolyte delivery system. Although aqueous solutions are preferred, the exact concentration of the salt solution is not critical, and in indeed, fully saturated salt solutions may be used. In some applications concentrated saturated saline solutions are preferred. However any solution of over 50% saturation may be suitably used. The solution may be conveniently prepared by simple addition of, for example, rock salt to a vessel such as a tank or holding device containing the water, or connected to a water supply. Mixing or solution preparation, filtration etc., are not usually necessary (unless input conditioning is required, for example if ionic salts of sufficiently high purity are not used). Sufficient rock salt should be provided so that a dilute solution of electrolyte is available at all times. The electrolyte delivery system may, for example, comprise a pumping device connected to the salt holding tank or other electrolyte supply or storage device. The skilled person will appreciate that any device capable of accurately delivering a measured amount of saline or electrolyte to the system may suitably be used. The delivery system is under management of and responds to commands from the control system.

In another embodiment, a measuring device/arrangement capable of measuring the volume or height (level) of electrolyte in the cell may serve as an indicator of the cell efficiency, since the electrolyte volume (and consequently height or level in the cell) required to maintain current will increase as cell efficiency decreases. As cell efficiency decreases, the volume of fresh electrolyte to be added to compensate for efficiency loss will gradually increase over time. The system may be set up to initiate a warning when a predetermined high volume of electrolyte is required to maintain cell current so that the cell operates at an acceptable efficiency level. The system can be calibrated to account for temperature and catholyte flow effects in the cell and their effect on anode liquid level. Changes in volume/level resulting from such parameters will not represent changes in efficiency and must be accounted for appropriated. Such methods will be known to the person skilled in the art. A similar system may be operated based on anode gas pressure.

In one embodiment, a preferred arrangement involves a water mains supply connected so as to deliver water to an electrolyte storage tank, with which the electrolyte delivery system is associated. This is an advantageous arrangement, since direct connection to the main supply is expected to provide a steady, reliable and generally un-interrupted source of water to the delivery system and will mean that manual filling of the electrolyte tank or manual electrolyte solution preparation will not be required. The current detecting system, under management of the control system, is in communication with the electrolyte delivery system. The information regarding the current provided to the control systems is used to calculate the additional electrolyte to be input by the electrolyte delivery system, so that the generated output solution of the electrochemical cell has a substantially constant concentration (reflecting substantially constant cell current). When the optimal current level has been restored (as indicated by the current detection system data) the control system may signal electrolyte input interruption and the electrolyte delivery system will cease electrolyte input until further adjustment is required.

The electrolyte delivery system may suitably be a volume measurement device, for example, any pump capable of accurately measuring the small volumes required to make adjustments to the cell current. It is important to note that the operation involves essentially continuous monitoring over defined or predetermined intervals of time and substantially continuous current adjustment by accurate electrolyte input dosing as required, so that in essence the current is kept virtually constant.

Advantageously, such an automated constant monitoring system based on current detection, avoids the existing electrochemical cyclical current change profile associated with crude prior art devices based on relatively inaccurate level monitoring and thus ensures a more stable progressing cell current and thereby produces a more stable and consistent biocidal output that was previously achieved.

In a related embodiment, the electrochemical device may further comprise means for shutting down the electrolyte delivery system when the input volume of fresh electrolyte required to maintain the steady state current, has reached a predetermined level (which indicates a critical loss of efficiency). Typically, such a system will comprise a shut off valve or switch or any such system capable of shutting off the electrolyte delivery system and/or current and/or voltage across the cell, to the effect that electrolysis ceases. Such an arrangement is advantageous, since over a period of time, the cell efficiency will steadily decrease until such time that as the output does not meet the required standard. To compensate for loss of efficiency, as time progresses, more and more electrolyte will be required to maintain the constant cell current. A system that shuts down at a predetermined point of efficiency loss is desirable, since the cell can be serviced or repaired before the output solution standard falls below a particular quality. This is advantageous, as it avoids inadvertent production of sub-standard biocidal solution.

In yet another related embodiment, the electrochemical device may also comprise an alerting means for indicating that the input volume has substantially reached or is approaching the predetermined level (or that gas pressure has reached a certain level). Suitably, such a system may comprise a warning light or an alarm warning system, such as sound alerting means or the like. Specific advantages arise from this system, since the operator will be warned in advance that a point of critical loss of efficiency is imminent and will be prepared to service the cell to reduce production downtime. In other words, the system will give advance warning that the critical loss of efficiency point is approaching. Thus, the invention provides an improved automated electrochemical device that produces warning messages, signals and/or sounds to indicate that the cell efficiency has fallen to a preset degree and that the cell or system may require attention. This aspect of the invention ensures that electrochemical device is operating within desired parameters so as to guarantee the safe and efficient operation of the device and consistent production of output solution.

According to the present invention, there is also provided an automated electrochemical device for generating a biocidal output solution, said device comprising:

a flow-through electrochemical cell comprising an anodic chamber and a cathodic chamber for electrolysing an electrolyte to generate an anolyte solution and a catholyte solution, characterised in the device further comprises:

(i) a reservoir for storing catholyte; and

(ii) a hydraulic circuit for recirculating catholyte from the reservoir to the anolyte on start-up of the cell,

wherein input of catholyte of a compensating strength to the cell anodic chamber, is arranged to optimise the cell anolyte pH to and produce a stable output solution at the start of the electrolysis process.

By the term “catholyte of compensating pH”, it is meant that catholyte solution will be of suitable pH (basic) to effect the desired pH change required for anolyte adjustment (in other words to stabilize the biocidal output). For example, if the anolyte is too acidic due to the presence of excess hydrogen ions in solution, a compensating catholyte pH will be one which is basic enough to lower the acidic pH of the anolyte to a move favourable value. When the anolyte is at the optimum pH, the equilibrium species will be in the correct concentration to ensure consistent biocidal properties. The system of the invention is advantageous over the prior art catholyte recirculation systems, since the initial catholyte produced in such electrochemical cells is low in hydroxide ion until electrolysis has progressed for some time and hydroxide ions have had sufficient time to accumulate to provide a catholyte solution having a low enough pH to be compensating. Prior art systems are designed to then recirculate the optimised catholyte once the desirable catholyte pH conditions have been achieved. It remains the case that a certain period of time to produce optimised catholyte for recirculation is required and accordingly start up times are delayed. For discussion of the features described herein, it is clear that the pH of the anolyte solutions is a critical parameter and will have a fundamental effect on the solution species “free chlorine” equilibrium concentrations and states. Thus, a means for conveniently adjusting and regulating pH of the anolyte solutions is advantageous. Thus, a device capable of storing and recirculating existing “readymade” compensating catholyte solutions is attractive and desirable, since it obviates the need to wait for optimisation of catholyte for recirculation, since pre-optimised catholyte is stored and is ready for use immediately when the device is started. This means that the intial output is pH stablized more quickly and less product is wasted. Furthermore, the system of the invention obviates the need to drain the catholyte from the electrochemical device. Prior art systems must be drained after use to avoid damage due to the corrosive nature of the optimised catholyte. Advantageously, processing time are reduced. Thus, the invention is advantageous over existing systems since it automatically overcomes the existing undesirable prolonged period of unstable pH at start up of an electrochemical generating device which results in inconsistent output production. Furthermore, the invention allows for automated control of the output product pH during normal operation of the electrochemical device so as to ensure a consistent, less wasteful output solution. Advantageously, the invention reduces start up time. Another advantage of such a system is that wasteful initial outputs are avoided or are at least minimised to a more acceptable level.

In one embodiment, the reservoir may be external to the electrochemical device, e.g., the reservoir may take the form of an externally located tank or other storage device, from which catholyte may be supplied to the cell anolyte as required, through an appropriately connected input line. Preferably, the reservoir is made from a corrosion resistant material, as the stored optimised catholyte solutions will be corrosive. It will be appreciated that in this arrangement, a suitable catholyte substitute solution such as sodium hydroxide or potassium hydroxide may be used to optimise the anolyte solution. In other words, a catholyte solution which is not generated by the system itself may be used. However, it is preferable that the reservoir be located inside the device. It is also preferred that catholyte produced by the system itself be is used in anolyte optimisation by circulation of pre-optimised catholyte. Specific advantageous arise from an internal arrangement of the resorvoir, since the device is neater and contamination from outside sources is less likely to occur and cause problems during device operation. Having the reservoir in the internal arrangement obviates the need for use of external storage containers or facilities to store the catholyte until the next generation cycle is initiated. Additionally, labour involved in preparing suitable sodium hydroxide or potassium hydroxide solutions is avoided when system generated pre-optimised catholyte is used.

The electrochemical device hydraulic circuit and/or the reservoir further comprises at least one drain. This is advantageous since it facilitates removal of aged catholyte and allows easier cleaning and/or maintenance of the system, as required.

The invention further provides an automated electrochemical device for generating a biocidal output solution, said device comprising:

a flow-through electrochemical cell for electrolysing an electrolyte to generate an anolyte composition and a catholyte solution;

characterised in that the device further comprises:

a pH-regulating system for adjusting the pH of the output solution,

whereby dosing the catholyte solution into the anolyte solution based on the amount of catholyte solution required, effects a desired output solution pH adjustment.

The skilled person will appreciate that the anolyte composition may be of gas, solution, aerosol or combinations thereof.

In a related embodiment, there is provide a method of producing a consistent biocidal solution from an electrochemical cell comprising the steps of

(i) electrolysing an electrolyte to produce a catholyte and an anolyte solution;

(ii) adjusting the pH of the catholyte to a desired level;

(iii) dosing a predetermined amount of catholyte into the anolyte

to produce an anolyte output having a predetermined pH.

The catholyte can be mixed with the output solutions of the device in order to regulate the pH to a desired level or to dilute the anolyte if and when it becomes too concentrated. As discussed earlier, the pH of the cell electrolyte solution is important to control the electrolysis products and in particular propensity towards chlorine gas evolution. Discrete control and stabilisation of the anolyte and final output solution pH and concentration is key to providing consistent product biocidal outputs having reliable biocidal activity.

The electrochemical device thus has a hydraulic circuit suitable for supplying catholyte to either or both of the anolyte solution and to the final biocidal output solutions. This catholyte hydraulic circuit may also comprise a pH meter and water supply source (catholyte dilution system) for adjusting the concentration of the catholyte before it is dosed into the anolyte solution. This is advantageous insofar as it assists in the production of consistent outputs of the correct concentration.

Thus, the electrochemical device hydraulic circuit may also comprise a pH-regulating system in the catholyte hydraulic circuit. A suitable system is a catholyte pH-regulating device. Suitably, the catholyte pH-regulating control device may comprise a pH meter and solution mixing/dilution device connected to a fresh water supply for adjusting the concentration of the catholyte before it is dosed into the anolyte solution. The pH meter measures the pH of the catholyte, relays the information to the control system and the control system will calculate the amount of dilution required to provide a catholyte solution of required strength to regulate the output solution and provide that data to the catholyte mixing/dilution device. This allows finer control of the adjustments that can be made to the anolyte, since the catholyte dose itself is adjustable through a dilution step, if necessary. The catholyte pH regulation system is advantageous insofar as it assists in the production of consistent outputs of the correct concentration and pH by ensuring the catholyte to be dosed into the anolyte is of a suitable pH and concentration.

In another embodiment, the device also comprises a final biocidal output pH-regulating control device that comprises a pH meter and water supply source (output solution mixing/dilution system) under management of the control system. In some arrangements, a hydraulic line may be provided to the final output line to allow catholyte to be dosed into the output, if required. The pH-regulating control device is preferably located downstream of the final output hydraulic circuit and is used to make final adjustments to the biocidal output solution after the initial catholyte dosing step. Thus, the final biocidal output solution pH properties or strength may be determined by the final biocidal output pH-regulating control device. Thus, the invention also provides an improved automated electrochemical device, and a hydraulic system for use in such a device, that facilitates production of a consistent output solution by allowing dilution and pH regulation of the output solution before and/or after initial pH adjustment by catholyte dosing. Thus, the electrochemical device thus has a hydraulic circuit suitable for supplying catholyte to the anolyte and also, if necessary, to the final biocidal output solution.

In one particular embodiment, the catholyte supply may be provided to the catholyte hydraulic circuit from a reservoir that is external to the electrochemical device. For example, the reservoir may take the form of an externally located tank or other storage device from which catholyte may be supplied to the cell anolyte, as required. It will be appreciated that in this embodiment, a suitable catholyte substitute solution such as sodium hydroxide or potassium hydroxide could be used to optimise the anolyte and/or output solution. However, it is preferable that such a reservoir be located inside the device and that catholyte produced by the system itself be used in anolyte and/or output optimisation. Specific advantages arise from an internal arrangement, since the device is neater and contamination from outside sources is less likely to occur and cause problems during device operation. Additionally, labour involved in preparing suitable sodium hydroxide or potassium hydroxide solutions is avoided.

The device dilution systems may comprise a pump or the like, capable of delivering a predetermined amount of diluent accurately. Alternatively, the delivery system may comprise an arrangement where water is simply added until the desired pH is attained. The mixing/dilution feature(s) are advantageous insofar as they assist in the production of consistent outputs of the correct final concentration and pH. Thus, the final biocide output pH can be continuously and automatically adjusted during operation of the device by continuously dosing catholyte into the output solution until the desired output pH is produced and/or dilution of the biocidal output to dilute the output to the desired strength.

The invention provides an improved automated electrochemical device, and a hydraulic system for use in such a device, and a method of using same, which allows separation and diversion of the main supply solutions and the main supply required for the core electrolyte generation from the large volume main supply solutions required for the device, thereby allowing for the high level conditioning of the reduced volume core solutions at a lower cost and higher conditioning efficiency.

Overall, the entire device is generally operable by the control system to which information is relayed by the current detecting system (and system pH regulation devices and gas regulation components etc, if installed). The control system uses data sent by the system modules to provide information/instructions to the electrolyte delivery system, the anode gas pressure device, the catholyte mixing/ dilution device (catholyte pH regulation control device) and output pH-regulating control device.

It will be appreciated that the control system may be any processor device or electronic chip, circuit board or computing device set up to be capable of calculating the amount of additional electrolyte required to be input to maintain a steady state current in the cell and is further capable of providing start and cease instructions to the electrolyte delivery system. The control system must also be capable of calculating pH and dilution requirements and delivering “start” and “ cease” instructions to the device modules such as including the anolyte/catholyte and/ or output mixing and dilution systems. Suitably, the device is a computer or electronic circuit board.

The present invention also relates to the provision of a system and a method of providing a reduced volume stream of pre-conditioned input solutions to the electrochemical cell, so as to ensure a more consistent supply of output and to avoid cell downtime resulting from mineral deposit contamination of the electrochemical apparatus. In this aspect, the invention discloses a method whereby input conditioning can be carried out in an economical and convenient manner. In general, the amount of solution required for the core generating process is only a very small proportion of the volume of solution that is output from the device. The low volume of solution required in the core generating process allows for the high standard conditioning of these solutions resulting in a more stable and defined output to the generating device. Thus, the invention provides a system and a method whereby the generating input solutions to be conditioned are maintained in a separate hydraulic circuit to those for the general output solutions of the device at a greatly reduced volume when compared to the mains supply. Introduction of uniform core generating solutions into the cell, results in uniform output from the device. Beneficially, the maintenance and cleaning of the device can be reduced due to the fact that the input solutions are of a high standard without contaminants present.

In a different aspect, the device may be used on its own to generate biocidal solutions, which may be collected in a storage vessel or tanker until required for use in the particular application of interest or for further processing or product packaging, etc.

In a different aspect, the device final output hydraulic line may be adapted to be interfaced interface directly with the system or area to be treated with the biocidal solution. For example, the device may be installed near the water systems of air conditioning units or the like, or near a building's water heating systems, for example, hospital water systems. This arrangement has the advantage that the device output is directly feed into the system to be treated. The device may be set up so that output is supplied to the system to be treated at a suitable flow rate, for a suitable period of time. This has further advantage since personnel will not be required to manually use the biocide to treat the system in question.

With reference to the various specific embodiments described herein, it is important to point out that particular advantages arise from combining one or more features of any of the embodiments of the above invention. Combinations of one or more of the feature is possible and provides optimised biocidal products. Any particular combination of the features as set out in the claims and in the description is possible and specific combinations will provide particular advantages. In particular, it will be appreciated that a device of the invention can incorporate any combination of the features described. While the present inventors have made many independent improvements, it will be appreciated that each of the improvements can be used in combination with any of the others, particular those features mentioned independently. It is particularly advantageous for example, to combine the cell current stabilisation feature with the reduced startup time features and further advantageous to combine either of these features alone or in combination with the automated system efficiency measurement and adjustment features and in further combination with the automated system pressure detection and control feature, and/or the output dilution feature or any sub-combination or permutation of the independent features.

### BRIEF DESCRIPTION OF THE DRAWINGS

The invention will be more clearly understood from the following description of an embodiment thereof, given by way of example only, with reference to the accompanying drawings, in which:—

FIG. 1 shows a schematic drawing of the typical hydraulics and components of an electrochemical biocide generator of the invention.

FIG. 2 shows a schematic drawing of the hydraulics and components of an electrochemical biocide generator of the invention having an optional evolved gas pressure meter.

FIG. 3 shows a schematic drawing of the hydraulics and components of an electrochemical biocide generator of the invention having a water conditioning system.

### DETAILED DESCRIPTION OF THE INVENTION

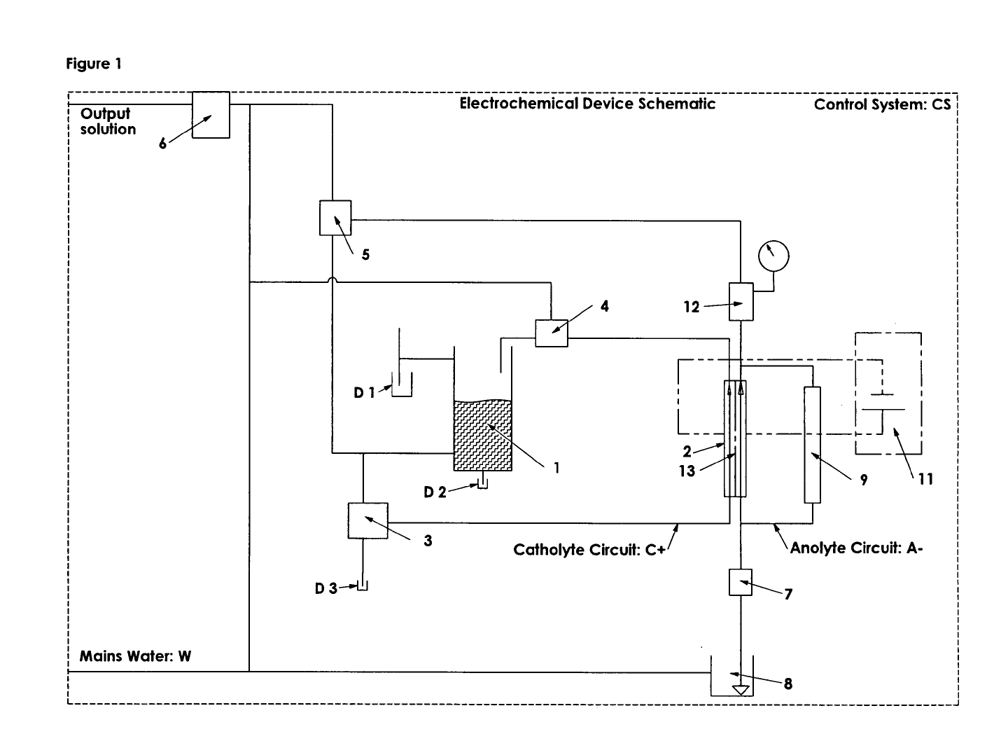

This invention relates to an electrochemical device designed to produce antimicrobial solutions. Referring now to the drawings and specifically FIGS. 1 to 3 inclusive and initially FIG. 1.

FIG. 1 shows an electrochemical device of the invention. The device is operable under the instruction of the control system CS (represented by the dashed rectangle in the figures). The device comprises two distinct hydraulic circuits, a catholyte circuit and an anolyte circuit represented by C⁺ and A⁻ respectively, which feed:

(i) electrolyte input to, and anolyte output from, the anode chamber of the electrochemical cell 2 along anolyte hydraulic circuit A⁻ so that a gaseous product (composed of chlorine in the main) is formed therein; and

(ii) re-circulating catholyte input to the electrochemical cell 2 and catholyte output from the cathode chamber of the cell 2, wherein hydroxide ion is produced, to the catholyte storage device 1 and to the catholyte pH regulation control device 4 along circuit C⁺.

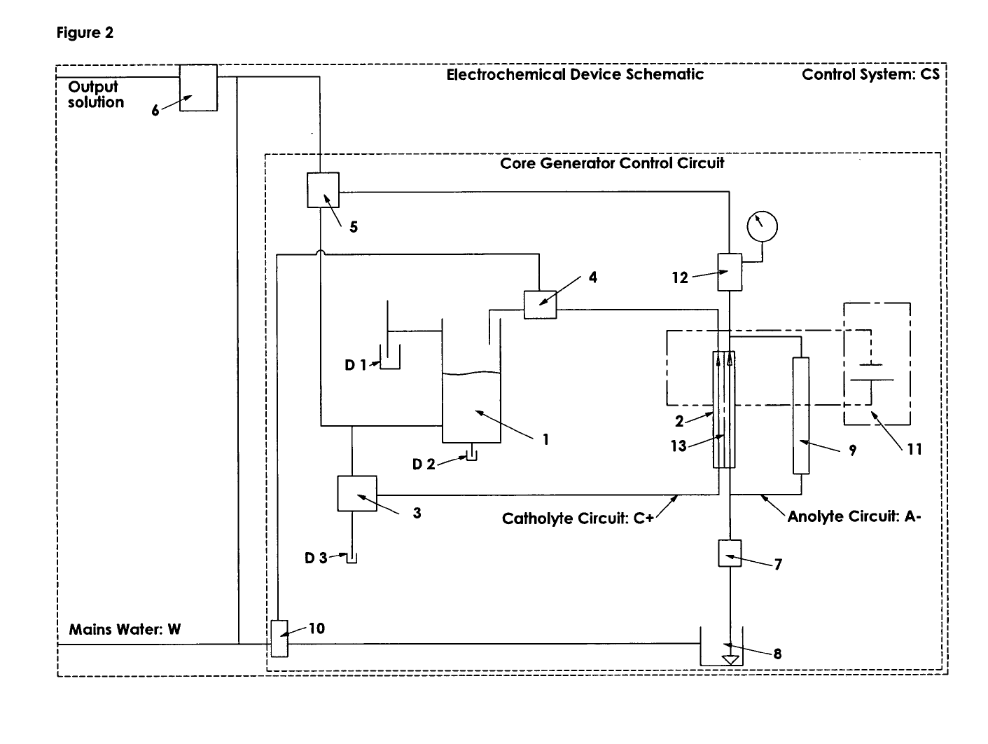

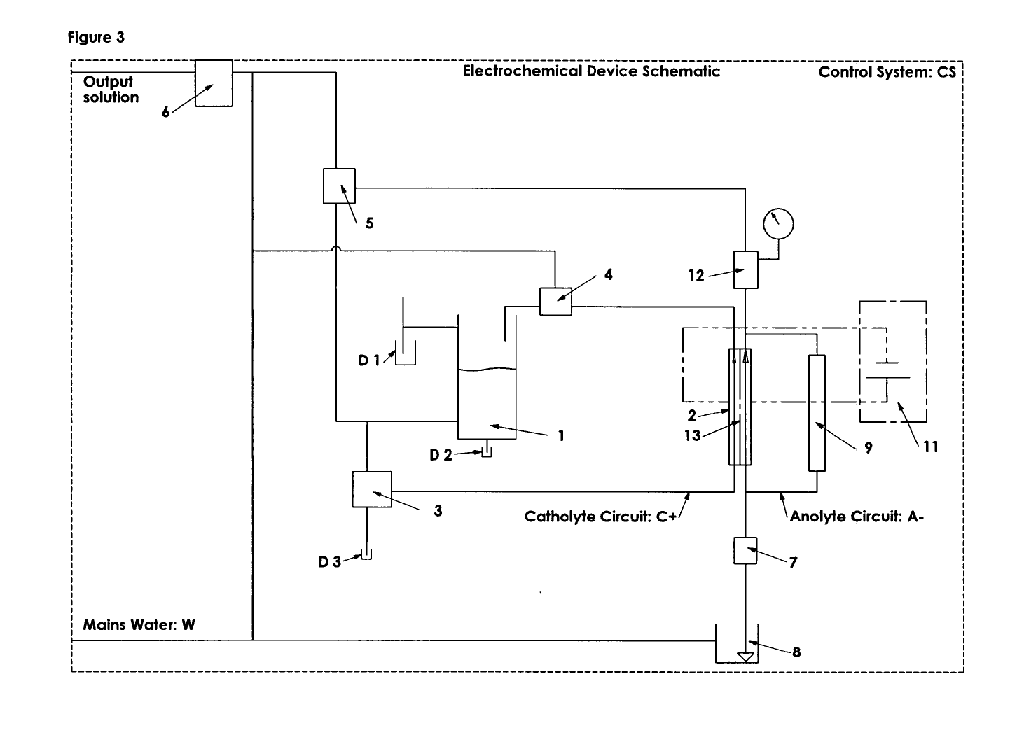

Hydraulic circuit A⁻ can be optionally connected to an electrolyte volume/level indicating device 9, which can be calibrated to indicate losses in efficiency and to account for flow/temperature effects on electrolyte level. The volume/level indicating device 9 simply provides a reading of level or height of electrolyte in the anode chamber. A current detection system 11 is electrically connected to the electrochemical cell 2 and to the volume/level detection device 9. FIGS. 2 and 3 show an evolved gas pressure measuring device 12, which capable of adjusting gass pressure at the anode, is connected to the electrochemical cell 2 to provide data to the control system CS regarding gas pressure at the electrodes in the cell 2. The evolved gas pressure measuring device 12 allows control of the salt content in the output stream and provides an indication of the current passing through cell 2. Gas pressure measuring device 12 is shown in schematically FIGS. 2 and 3, positioned along the output stream between the electrochemical cell 2 and the anolyte pH regulation control device 5 along hydraulic circuit A⁻. Gas pressure measuring device 12 is also capable of adjusting the gas pressure at the anode.

The system comprises a water input hydraulic system that delivers a mains water supply W, which is designed to provide water to (i) the electrolyte storage tank 8 and electrolyte delivery system 7, (ii) the catholyte storage 1 and hydraulic circulatory system C⁺ and to (iii) a pH measuring device/dilution system 6 positioned on the output stream downstream of the anolyte pH-regulating control device 5 and catholyte pH-regulating control device 4.

The hydraulic circuits C⁺ and A⁻ are directly isolated from each other by the electrochemical cell ion permeable membrane 13 which allow separation of the solutions ions according to charge when a current is applied across the electrochemical cell 2.

Hydraulic system C⁺, further comprises a catholyte pH-regulating control device 4, a startup catholyte circulation and drain device 3 which allows recirculation of catholyte during electrolysis, and drainage valves D1, D2 and D3 to drain solution from (i) startup catholyte circulation and drain device 3, (ii) the catholyte storage device 1 and (iii) overflow from the catholyte storage device 1, respectively. The catholyte pH regulation control device 4, the anolyte pH regulation control device 5, the output pH regulation control device 6 and the start-up catholyte circulation device 3 may be separately connected to the main supply so that fresh water is available, if required for catholyte dilution and mixing.

FIG. 2 shows water hydraulic system W, connected to a water conditioning unit 10 which may be positioned on the mains input stream before the divergence to input, dilution and pH measuring streams. The catholyte pH regulation control device 4 is connected to the conditioned water supply leaving the water-conditioning unit 10 in FIG. 2. In operation, as the current detection system 11 indicates that the electrochemical cell current is rising, the amount of saline solution being discharged by the electrolyte delivery system 7 under influence of the control system CS into the cell is reduced, such as to ensure current output is maintained at a predetermined level. As the electrolysis process proceeds and the current detection system 11 is activated to determine the current over a predetermined period of time. If a current reduction is indicated, the amount of saline solution being delivered into cell 2 by the electrolyte delivery system 7 is increased to a required level to re-stabilise current output. The process is repeated and results in steadying the current in the cell to a substantially constant level as necessary. It is important to note that the operation involves essentially continuous monitoring over a predetermined time interval and substantially continuous current adjustment so that in essence the current is kept substantially constant. Saline input can be set up to occur at discrete intervals at fixed flow rates, for a fixed time period. In this case, when the current drops below the set value the electrolyte delivery system 7 inputs saline into cell 2 as the electrolyte delivery system 7 is activated to deliver for a fixed period of time at a fixed delivery speed. Thus a defined volume of saline solution is inputted into the cell 2. If the current is above the set point after this delivery period then the electrolyte delivery system 7 is not activated again and no more solution is inputted into cell 2. If however the current is still below the set point after this delivery period the electrolyte delivery system 7 is reactivated and the process is continued until the desired current is achieved.

During the electrolysis process, saline solution is inputted into the electrochemical cell 2 though an electrolyte delivery system 7 and a voltage/current is applied to the cell 2 to commence electrolysis. Since the overall biocide component output from the electrochemical device is a function of the amount of current passing through the device, the output solution from the device can be maintained at a desired level or state, if the current can be maintained at a particular predetermined level. The present system operates optimally because current is monitored continuously over set intervals and adjustment is made, so that a substantially constant electrical current flows across cell 2. Thus, the output from the device can be controlled and altered to any desired level by controlling and adjusting the level of saline solution in cell 2. Saline level in the cell 2 has an effect on the cell current because the level of saline present determines the portion of the anode wetted by the liquid and able to oxidise chloride to chlorine. Any alterations in cell current that occur as electrolyte consumption proceeds (indicated by electrolyte level dropping) may be compensated for by input of fresh saline electrolyte solution. The compensating volume needed will be dependent on the degree of current compensation required. Advantageously, such an automated constant monitoring system avoids the existing crude electrochemical cyclical current change profile and thus ensures a more stable progressing cell current than prior art systems based on electrolyte level monitoring only. A more stable and consistent biocide component output results. As the cell volume/level of electrolyte solution as indicated by electrolyte volume/level indicating device 9 increases for the maintenance of a given current, this indicates that the efficiency of the device is decreasing. The current detection system 11 measures the current in cell 2 and the electrolyte volume/level indicating device 9 measures the cell solution volume and/or solution level. The two measurements are then compared and the efficiency can be determined. If the device is about to reach a critical state of lost efficiency and is about to suffer a failure as a consequence, the volume/level measurement to current measurement ratio will change. This can be detected and the user is therefore forewarned and remedial action can be taken. For example, a warning can be initiated or a cleaning process can be triggered or a shutdown procedure can be initiated. The system can be calibrated to account for temperature and catholyte flow effects in the cell and their effect on anode liquid level. It should be noted that changes in volume/level resulting from such parameters will not represent changes in efficiency and must be accounted for appropriated.

The cell current and/or efficiency of cell 2 is monitored by gas pressure-measuring device 12 on the anolyte hydraulic stream A⁻ as shown in FIGS. 2 and 3. The amount of chlorine gas produced and thus the chlorine gas pressure produced for a particular cell current is indicative of the cell efficiency. A drop in gas pressure as indicated by gas pressure-measuring device 12 shows that current in cell 2 is falling or that the current in cell 2 is still constant, but that the cell efficiency is falling. When a critical predetermined point is reached, this is indicative that cell 2 may require attention. At device start-up the strength of the catholyte is often of insufficient ionic conductivity or pH to generate the operating currents desired or regulate the pH of the output biocide. High strength catholyte may be stored external to cell 2 in storage reservoir 1 (shown in FIGS. 1 to 3) when the device is inactive so that this catholyte may be circulated through the cell at device start-up to reduce device start-up times. Referring now specifically to FIG. 2, the vessel is hydraulically connected to a startup circulation and drainage device 3, the electrochemical cell (FEM) 2, and anolyte pH regulation control device 5. When the electrochemical device is stopped or is not in use, the catholyte from a previous operation of the device is pumped into and retained in the storage reservoir 1 or from an external reservoir or stock of previous catholyte or indeed operator prepared sodium hydroxide or potassium hydroxide solutions, depending on the configuration of the device. On system start up, the stored catholyte is discharged back into the cathode chamber of cell 2 to allow rapid establishment of the optimum operational cell 2. The catholyte solution can also be delivered to the anolyte or device output solutions by the anolyte pH regulation control device 5, in order to create the desired biocide output pH. The electrochemical device can produce stable pH output almost immediately after the device has started up, since the initially introduced catholyte is of sufficient strength to produce the desired pH output immediately and hence normal operating current can be achieved more quickly.

Carrier aqueous solution is passed through the device for mixing with the anolyte solution or the output solution to form the biocidal output solution of the desired pH. Measurement of the pH of the output solution allows the flow of the carrier aqueous solution to be regulated as required, so that changes in concentration and/or pH of the output solution can be made to provide stable and consistent biocide output from the device. Alternatively, if a gas is being produced in the electrochemical device, the volume of the gas produced will depend on the efficiency of the device. In this case, gas measurements can be used to assess the flow of the carrier aqueous solution needed to regulate the output efficiency of the generating device, hence producing a more stable output if required.

Dosing and pH adjustment can be controlled automatically using a catholyte pH regulation control device 4 (FIGS. 1 to 3) to control the discharge volume of the catholyte. If the output pH drops below the desired level, the discharge of the catholyte from the storage reservoir 1 to the output stream can be increased. The actual concentration of the catholyte discharged does not need to be completely uniform, since the discharge rate can be varied to produce the desired output pH level.

This type of output pH regulation system is particularly useful since it has been found that the concentration of the re-circulating catholyte can be measured using pH measurement device 4. The pH of the catholyte can also be determined by measuring the volume of catholyte that is being added to the output solution in order to produce the specific pH value for a given flow of output. For example, catholyte of a low sodium hydroxide concentration may require a flow rate of 45 ml of catholyte per minute to be discharged into the output solution to produce pH 7.0 for a given flow rate. Catholyte of high sodium hydroxide concentration may require 30 ml of catholyte per minute in order to produce pH 7.0 of the same flow. Over time, the re-circulated catholyte becomes stronger, the pH rises and the solution becomes more caustic. Once the strength of the catholyte reaches a set level, the catholyte can be diluted in order to maintain the pH at a predetermined level. This is achieved by controlling the discharge of a dilute agent, which is generally water. The catholyte pH-regulating device (mixing/dilution device 4) is linked to the mains water supply W and to the output regulating device 6 (pH meter) along the output hydraulics circuit. Information from the output regulating device 6 (pH meter), together with output stream flow rate data, allows the level of anolyte dilution or catholyte input dosage to be calculated. Information regarding the pH of the output is sent to a control system CS, which determines whether concentration/pH adjustments are required and implements same. Thus the invention provide a device whereby the flow of the aqueous solution passing through the device is automatically detected and regulated based on detected efficiencies and the pH and concentration of the biocide application requirements.

Referring now to specifically to FIG. 3, a portion of the water from the mains water hydraulic system W, is diverted away from the main circuit supplying the output dilution stream, and is directed at a much-reduced volume into the water-conditioning unit 10. The conditioning unit 10, depending on its form, removes ions from the water and supplies conditioned water to the electrolyte storage tank 8, and the electrolyte delivery means 7, which is eventually supplied to the electrochemical cell 2 in conditioned form through the anolyte hydraulic circuit A⁻. The conditioned water is also supplied from the conditioner 10 to the catholyte recirculation hydraulic circuit C⁺. This ensures that any water reaching the cell, directly from the conditioner 10 or from the re-circulation circuit C⁺ is isolated from the untreated main supply and ensures more stable electrochemical processing in the cell and avoids cell downtime resulting from mineral deposits (due to calcium and magnesium hydroxides and carbonates and the like) in the cell.

Saline is referred to when other electrolytes such as other salts can be utilised.

The skilled person will appreciated that by use of the terms “anolyte solution”, it is mean that the solution is in fact a composition comprising a gas, a solution, an aerosol or any combinations thereof.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention that are, for brevity, described in the context of a single embodiment, may also be provided separately or in any suitable sub-combination.

The invention is not limited to the embodiments hereinbefore described but may be varied in both construction and detail.

1. An automated electrochemical device for generating a biocidal output solution, said device comprising:
a flow-through electrochemical cell comprising an anodic chamber and a cathodic chamber for electrolysing an electrolyte to generate an anolyte solution and a catholyte solution; characterised in that the device further comprises
(i) a reservoir for storing catholyte; and
(ii) a hydraulic circuit for recirculating catholyte from the reservoir to the anolyte on start-up of the cell,
wherein input of catholyte of a compensating strength to the cell anodic chamber, is arranged so as to optimise the cell anolyte pH to produce a stable output solution at the start of the electrolysis process.

2. An electrochemical device according to claim 1 wherein the reservoir is external to the electrochemical device.

3. An electrochemical device according to claim 1 wherein the hydraulic circuit further comprises a catholyte pH regulation control device.

4. An electrochemical device according to claim 1 wherein the device further comprises an output pH regulation control device.

5. An automated electrochemical device for generating a biocidal output solution, said device comprising:
(i) a flow-through electrochemical cell for electrolysing an electrolyte to generate the output solution;
(ii) a current detection system connected to the electrochemical cell for determining when cell current reaches a predetermined level; and
(iii) an electrolyte delivery system operable by the current measuring system; characterised in that wherein said delivery system inputs a volume of electrolyte into the cell when a predetermined level of current is detected, so that the generated output solution of the electrochemical cell has a substantially constant concentration.

6. An electrochemical device according to claim 5 wherein the current detection system comprises a current measurement device.

7. An electrochemical device according to claim 5 further comprising means for shutting down the electrolyte delivery system when the input volume of input electrolyte has reached a predetermined level.

8. An electrochemical device according to claim 7 further comprising an alerting means for indicating that the input volume has substantially reached the predetermined level.

9. An automated electrochemical device for generating a biocidal output solution, said device comprising:
(i) a flow-through electrochemical cell for electrolysing an electrolyte to generate an anolyte solution and a catholyte solution;
(ii) a hydraulic circuit for supplying catholyte to the anolyte solution;
characterised in that the device further comprises:
a pH-regulating system for adjusting the pH of the output solution
whereby dosing the catholyte solution into the anolyte solution based on the amount of catholyte solution required, effects a desired output solution pH adjustment.

10. An electrochemical device according to claim 9 further comprising a reservoir for catholyte, which may be internal or external to the device.

11. An electrochemical device according to claim 9 wherein the hydraulic circuit further comprises a catholyte dilution system for adjusting the concentration of the catholyte before it is dosed into the anolyte solution.

12. An electrochemical device according to claim 9 further comprising a biocidal output dilution system for adjusting the concentration of the biocidal output solution after pH adjustment.

13-19. (canceled)

20. A biocidal solution produced by the electrochemical device of claim 1.
